(19) United States
(12) Patent Application Publication
(10) Pub. No.: US 2019/0151192 A1
(43) Pub. Date: May 23, 2019

(54) ULTRASONIC IRRADIATION APPARATUS AND SYSTEM, AND ULTRASONIC IRRADIATION METHOD

(71) Applicants: Yohachi YAMASHITA, Yokohama-shi, Kanagawa (JP); KAIKAI CO., LTD., Imizu-shi, Toyama (JP)

(72) Inventor: Yohachi YAMASHITA, Kanagawa (JP)

(73) Assignees: Yohachi YAMASHITA, Yokohama-shi, Kanagawa (JP); KAIKAI CO., LTD., Imizu-shi, Toyama (JP)

(21) Appl. No.: 16/302,611

(22) PCT Filed: May 8, 2017
(86) PCT No.: PCT/JP2017/017407

§ 371 Date: Nov. 16, 2018

(30) Foreign Application Priority Data
May 19, 2016 (JP) 2016-100822

(51) Int. Cl. A61H 33/00; A61M 21/02; A61H 33/02; A61N 7/00

(52) U.S. Cl. CPC A61H 33/0091; A61M 21/02; A61M 2021/0027; A61N 7/00; A61N 2007/0095; A61H 33/02

(57) ABSTRACT

An ultrasonic irradiation apparatus includes an ultrasonic resonator capable of generating ultrasound, a driving unit configured to drive the ultrasonic resonator, a case holding the ultrasonic resonator and the driving unit, an acoustic matching layer provided between the ultrasonic resonator and the case, and an acoustic diffusion layer made of an ultrasound diffusing material configured to diffuse high intensity ultrasound emitted from the ultrasonic resonator and convert the high intensity ultrasound into low intensity ultrasound with low intensity per unit area and radiate in a large area. The ultrasonic resonator is a piezoelectric resonator, the ultrasound diffusing material of the acoustic diffusion layer is made of a metal material of an acoustic impedance of at least 40, and the acoustic diffusion layer is provided in at least one of inside of the acoustic matching layer, inside of the case, and outside of the case.

### TECHNICAL FIELD

The present invention relates to an ultrasonic irradiation apparatus, an ultrasonic irradiation system, and an ultrasonic irradiation method capable of providing ultrasound stimulation to bones of desired part or bones of almost the whole body excluding the head of human or mammals in order to improve body functions of human or mammals (mammals here include horses, cows, dogs, cats, or the like other than human).

### BACKGROUND ART

The world population will be 7,300 million in 2015, and population of elderly people aged 65 and over will reach 600 million in the world, occupying 8.2% of the total population, and increasing every year. One of large social issues caused by the increase in population of elderly people is cost increase in medical care, nursing care, and other welfare related services. Therefore, maintaining and improving health of elderly people at low cost are now in great social need.

Conventional health care methods are based on precision diagnosis of Western medicine, and main treatment methods are medication and operations of affected parts. However, diagnosis, inspection, operations, treatment, and so forth using advanced apparatuses are high in cost, so that not all the people can get sufficient treatment in developing countries.

In Eastern medicine, an entire treatment such as acupuncture, moxa cautery, and so forth in which the energy called “qi” in the whole body is made to enhance is mainly used. However, Eastern medicine also depends on special techniques (acupuncture and moxibustion and so forth) conducted by specialists, and there is no inexpensive health care apparatus or method which can be implemented generally and easily at home. Therefore, it has been difficult to implement a health care apparatus or a health care system with a simple apparatus at a low cost at home only by conventional Western medicine and Eastern medicine therapeutic methods.

Many reports have been made about treatment and health care using sound waves or ultrasound. For example, Patent Literature 1 proposed by Duarte of University of Sao Paulo of Brazil discloses a method of providing low intensity pulse ultrasound (hereinafter, LIPUS) stimulation to a fractured part using a contacting ultrasound probe. According to the report, this method provides LIPUS stimulation to a fracture part and promotes treatment of the fracture. The frequency of the ultrasound probe is 1.5 MHz at a period of 0.67 μs, the pulse repetition frequency (hereinafter, PRF) is 1,000 Hz (a period of 1 ms), the ultrasound duty factor is 20%, and the intensity spatial average temporal average (hereinafter, Isata) is 30 mW/cm² to 60 mW/cm². The duty factor is a ratio of the time in which ultrasound is actually transmitted among total time. For example, if 200 μs is transmitted and 800 μs stopped, the duty factor is 20%. According to the report, the LIPUS stimulation is provided for 20 min/day, 5 or 6 times/week, continued 3 to 10 weeks so that treatment of fracture is promoted.

A health care apparatus of Patent Literature 2 discloses an apparatus which generates great physical force under the water by injecting compressed air and water from a narrow nozzle, and provides stimulation to the whole body excluding head in a tank using bubble breaking energy. Patent Literature 3 discloses a wave motion beauty device which improves a facial treatment effect and a body slimming effect using mechanical vibration and electromagnetism radiation. Patent Literature 4 discloses an ultrasound bath capable of improving a relaxation effect by applying ultrasound to a human body in a bathtub, modulating the ultrasound to change sound intensity and outputting music. Patent Literature 5 discloses an apparatus which irradiates an affected part and so forth with ultrasound using a contacting ultrasound probe to conduct treatment. According to the report, stimulation is provided in this apparatus with the frequency of 0.1 MHz to 10 MHz, the pulse width of intermittent repetition pulse of 1 μs to 500 ms (frequency of 1 MHz to 2 Hz), the PRF of 1 Hz to 100 Hz, and the ultrasound intensity of 10 mW/cm² to 60 W/cm². Patent Literature 6 discloses an apparatus which achieves weight loss by irradiating the abdomen and so forth with ultrasound using a contacting ultrasound probe, and causes lipolysis by a thermal effect.

Patent Literature 7 discloses an ultrasound apparatus for arthritis treatment using ultrasound stimulation. Patent Literature 8 discloses a method for increasing bone mass of the leg by irradiating the heel and buttock of a user in a standing position or a seating position with ultrasound which is built into a board face of a case or a toilet seat. Patent Literature 9 discloses an ultrasound therapeutic apparatus which generates three different frequencies, 1 MHz, 2 MHz, and 3 MHz, from the same resonator. Patent Literature 10 discloses an apparatus which emits a plurality of low frequency ultrasounds of equal to or less than 1 MHz and, at a target part, a medium intensity pulse ultrasound of 50 mW/cm² to 450 mW/cm² as an ultrasonic apparatus which adjusts activity of a specific nerve cell.

Moreover, Non-Patent Literature 1 reports pain reduction of knee osteoarthritis and improvement in walking speed by LIPUS irradiation at an affected part of the knee. In this paper, the effects of LIPUS treatment on 140 patients with knee osteoarthritis are investigated. According to the report, the patients undergone the LIPUS treatment of the method made progress in all of the indices of knee pain, a bending angle of the knee, and 20-m walking speed, and the effect continues after one year of the investigation. The ultrasound apparatus used is related to the method of stimulating a periphery of the knee which is the affected part using a contacting ultrasound probe.

Non-Patent Literature 2 reports that an animal test about LIPUS is performed using ovariectomized mice, and that bone mass has increased and prevention of osteoporosis can be expected by LIPUS irradiation.

### CITATION LIST

### Patent Literature

Patent Literature 1: U.S. Pat. No. 4,530,360

Patent Literature 2: JP-A-2004-089474

Patent Literature 3: JP-A-9-276354

Patent Literature 4: JP-A-10-328056

Patent Literature 5: JP-A-2002-613

Patent Literature 6: Japanese Unexamined Patent Application

Publication (Translation of PCT Application) No. 2007-520307

Patent Literature 7: Japanese Unexamined Patent Application

Publication (Translation of PCT Application) No. 2008-514338

Patent Literature 8: JP-A-2015-36045

Patent Literature 9: U.S. Pat. No. 5,460,595

Patent Literature 10: Japanese Patent No. 5879402

### NON-PATENT LITERATURE

Non-Patent Literature 1: Mao-Hsiung Huang, Rei-Cheng Yang, Chia-Lee, Tien-Wen Chen, Ming-Cheng Wang Preliminary Results of Integrated Therapy for Patients With Knee Osteoarthritis, Arthritis & Rheumatism (Arthtis Care & Research) Vol. 53, No. 6, Dec. 15, 2005, pp 812-820

Non-Patent Literature 2: Dohyung Lim, et.al., Low-Intensity Ultrasound Stimulation Prevents Osteoporotic Bone Loss in Young Adult Ovariectomized Mice, J. Orthop. Res. 2011 January;29(1)116-25. doi:10.1002/jor.21191.

### SUMMARY OF THE INVENTION

### Problems to be Solved by the Invention

In the methods disclosed in Patent Literatures 1, 3, 5, 6, 7, 9 and 10 and Non-Patent Literatures 1 and 2 of the background art, the used ultrasound treatment probe is small in size, and the area to be simultaneously irradiated with ultrasound is limited to about 10 cm². These apparatuses provide partial stimulation to the body of human or mammals using ultrasound gel and so forth, and there is a problem that uniform LIPUS irradiation on almost the whole body or in a wide area is difficult.

In the injection method using bubbles disclosed in Patent Literature 2, the frequency of the generated ultrasound is limited to equal to or less than 100 kHz. It is an injection amount of bubbles that adjusts ultrasound intensity so that no particular apparatus for uniformly irradiating the ultrasound in the tank is used. There is a problem that this apparatus is stationary and as large as 10 kg or greater, and also noisy, so is not suitable for home use.

The method described in Patent Literature 4 provides ultrasound stimulation to a human body in a bathtub using an ultrasonic resonator installed on an outer surface of the bathtub. It is described that modulated pulse waves other than continuous waves are used to irradiate the human body with ultrasound of different intensities. In this method, since a highly directive ultrasonic beam focuses on a part of the body and thus may damage the body unless the human moves actively in the tank, only low intensity ultrasound can be used. Further, there is a problem that, in order to obtain high intensity (several tens of kW) ultrasound that is enough to vibrate the entire bathtub and wash the body, the apparatus becomes large. The expected effect of the ultrasound is body washing, and the effect of the music is only relaxation. Therefore, improvement in a physical function using LIPUS is not described at all.

Patent Literature 8 discloses a method for increasing bone mass by irradiating the heel and buttock of a user in a standing position or a seating position with ultrasound from an ultrasonic resonator built into a board face of a case or a toilet seat. However, no simple method for uniformly irradiating almost the whole body with LIPUS is disclosed.

Patent Literature 9 discloses an ultrasound therapeutic apparatus which generates three different frequencies, 1 MHz, 2 MHz, and 3 MHz, from the same resonator. However, this apparatus is large in size and is not suitable for home use.

Patent Literature 10 discloses an apparatus which emits a plurality of low frequency ultrasounds of equal to or less than 1 MHz and, at a target part, a low intensity pulse ultrasound of 50 mW/cm² to 450 mW/cm² or below as an ultrasonic apparatus which adjusts activity of a specific nerve cell. An object of this apparatus is to stimulate specific nerve cells in the brain and the heart, for example, and the apparatus is large in size and cannot be used easily at home.

Further, almost all of these apparatuses use an AC power supply of 100V to 220V as power supply, the human body is exposed to danger of current leakage all the time. Therefore, an ultrasonic irradiation apparatus of low voltage drive using a battery with improved safety is desired. In a battery-driven irradiation apparatus, it is necessary to use ultrasonic energy radiated from an ultrasonic resonator in the most efficient manner. However, since the ultrasound is highly directive for moving linearly, if ultrasound intensity is as high as several W/cm², for example, the body of human or mammals is exposed to danger unless the irradiated part is changed frequently. In an ultrasonic image diagnostic apparatus, a safety standard of the ultrasound intensity is defined to be equal to or less than 720 mW/cm² of Isata.

As described above, efficacy of ultrasound and LIPUS in the health care field is recognized well. However, conventionally known ultrasonic treatment instruments and illness therapeutic apparatuses using a LIPUS apparatus have various problems when used for home bathtubs containing 150 L to 1000 L of water. For example, an apparatus which generates bubbles from a side surface of a bathtub to massage the whole body or a part of the body has a frequency is less than 100 kHz, and there is still no report that such apparatus is effective in skeletal cell proliferation for fracture treatment or osteoporosis treatment.

Further, if an ultrasonic resonator and an ultrasound apparatus are used in a fixed manner to a side surface or a bottom surface of a bathtub, a toilet seat, and so forth, sound intensity of highly directive ultrasound cannot be made uniform. A technique of mechanical scanning and a beamforming for electronically bending a beam and so forth in order to change the direction of ultrasound is well known for the ultrasound medical diagnostic imaging apparatus. However, such a mechanism is complicated and expensive, and cannot be manufactured at a low cost.

A LIPUS apparatus used for healing bone fracture treatment or a portable ultrasonic beauty treatment device are small in ultrasound irradiation area of the piezoelectric resonator (equal to or less than 10 cm²). Therefore, in order to uniformly irradiate almost the whole body of an adult or a relatively large-sized mammal of which total surface area is 10,000 cm² or greater with LIPUS, 1,000 or more piezoelectric resonators are needed, which increases the apparatus size and the manufacturing cost.

Fiber reinforced plastic (hereinafter, FRP) which is a lightweight and highly heat insulating material generally used for home bathtub is made by kneading glass fiber and carbon fiber into epoxy resin. The acoustic impedance Z of an FRP bathtub is Z=3 MRayls to 6 MRayls based on loading weight of the fiber. Therefore, there is a problem that since a difference between the acoustic impedance Z of the FRP bathtub and that of water as a medium Z=1.46 MRayls is small, equal to or greater than 80% of ultrasonic energy applied to the FRP is absorbed and attenuated and lost in the FRP, so that irradiation efficiency for effective irradiation of the ultrasonic energy onto the human body is low.

As described above, an ultrasonic irradiation apparatus which is capable of uniformly providing low intensity ultrasound stimulation to almost the whole body excluding the head or a wide area of human or mammals using highly directive high intensive ultrasound, and which is simple in structure, affordable for the people in developing countries, and manufacturable at a low cost, a system using the apparatus, and an ultrasonic irradiation method are not currently known.

The present invention is proposed in consideration of these issues of the background art, and an object thereof is to provide an ultrasonic irradiation apparatus and a system, and an ultrasonic irradiation method capable of providing low intensity ultrasound stimulation to the whole body in a tank, such as a home bathtub or a relatively small-sized pool, as uniformly as possible using an apparatus of simple structure, low cost and small size.

### Means for Solving the Problem

The present invention includes an ultrasonic resonator capable of generating ultrasound, a driving unit configured to drive the ultrasonic resonator, and a case configured to hold the ultrasonic resonator and the driving unit. The ultrasonic irradiation apparatus includes an acoustic diffusion layer made of an ultrasound diffusing material on a surface of the case from which high intensity ultrasound (hereinafter, HIUS>1 W/cm²) is radiated by the ultrasonic resonator. The acoustic diffusion layer controls the HIUS to diffuse and scatter, and to partly transmit therethrough, converts the HIUS into low intensity ultrasound (hereinafter LIUS<60 mW/cm²) with low intensity per unit area, and radiate the low intensity ultrasound while controlling in a large area as uniformly as possible. Especially, the acoustic diffusion layer is provided at least one of inside of the acoustic matching layer, inside of the case, and outside of the case. The ultrasonic resonator desirably continuously and automatically generates a multi-high intensity ultrasound HIUS (several W/cm²) beam to human or mammals in at least two frequencies and two PRFs.

The ultrasonic resonator is a piezoelectric resonator which uses thickness vibration or radial vibration, and a resonance frequency thereof is equal to or greater than 0.3 MHz and equal to or less than 5 MHz. The piezoelectric resonator may be made of barium titanate (BT)-based ceramics, lead zirconate titanate (PZT)-based ceramics, lead magnesium niobate-based single crystal, a lead-free piezoelectric material, an organic piezoelectric material, CMUT (capacitive micromachined ultrasonic transducer), and so forth.

The ultrasound diffusing material of the acoustic diffusion layer is made of a metal material of an acoustic impedance of 40 or greater. Alternatively, the ultrasound diffusing material is made of foamed resin containing 90% to 99% or greater of air bubbles and gas. Especially this ultrasound diffusing material is made of a porous metal wire net of which opening is λ-λ/10 with respect to an underwater wavelength λ of the used ultrasound. The foamed resin is polystyrene foam or foaming polyurethane.

The ultrasonic resonator desirably is a lead-free piezoelectric material. Positions at which lead wires of the piezoelectric resonator and a power supply thereof are drawn desirably are separated from the ultrasonic resonator in the case and desirably located at an upper part when the apparatus is in use.

the ultrasonic resonator having at least one of two frequencies and two pulse repetition frequencies is placed in the case, and these continuously and automatically generate a plurality of ultrasounds when driven by the driving unit.

The driving unit including a power supply of the ultrasonic resonator and an ultrasonic oscillator including the ultrasonic resonator and the acoustic diffusion layer may be divided, the divided ultrasonic oscillator is waterproof and may be provided to be electrically connectable to the driving unit, and the divided ultrasonic oscillator may be connected to the driving unit, so that the ultrasonic resonator is operated.

Further, a holding member which is floatable on a water surface may be provided, and the case may be carried by the holding handle so as to be floatable and movable under the water surface.

Desirably, the acoustic matching layer of at least two types of materials is included, a thickness of the acousic matching layer is multiples of ¼ of an underwater wavelength λ of the used ultrasound, and a contour shape of the acoustic matching layer is 120% to 200% of the ultrasonic resonator in magnitude in a project area. A part of the acoustic matching layer is a transparent organic material which constitutes the case, and a thickness of the acoustic matching layer is odd number times ¼ of λ.

This ultrasonic irradiation apparatus desirably is a multi-ultrasonic irradiation apparatus in which an ultrasonic resonator which includes at least two frequencies and two PRFs is disposed in a single apparatus, a fundamental wave frequency of each of the ultrasonic resonators is in a range of 0.3 MHz to 5 MHz, and each of the ultrasonic resonators is continuously and automatically operated in series. A plurality of acoustic matching layers may be attached to the same piezoelectric resonator, the ultrasounds of a plurality of resonance frequencies are made to generate then, and ultrasound of at least two different frequencies may be generated by a single piezoelectric resonator.

At least two ultrasonic resonators are placed on a side surface of the case which is shaped as a pyramid, a cone, or a sphere, and each ultrasound radiation surface cross at an angle range of 60° to 200°. Further, Positions at which lead wires of the piezoelectric resonator and a power supply thereof are drawn desirably are separated from the ultrasonic resonator in the case and desirably located at an upper part of the case when the apparatus is in use. An ultrasonic resonator using a lead-free piezoelectric material is desirably employed.

The used ultrasound is a pulse wave, and a repetition frequency (PRF) is from 1000 Hz (1 ms) to 0.5 Hz (2 s), and a duty factor is 10% to 60%. The PRF includes at least two selected from 1 ms to 3 ms and 20 ms to 40 ms, and 500 ms to 2000 ms. Pulse wave intensity of this ultrasound may be reduced gradually from intermediate time of the start to completion. Besides this ultrasonic generator, a device which generates arbitrarily selected music as audible sound may be used. This ultrasonic irradiation apparatus may be rechargeable battery-driven apparatus, and is a waterproof ultrasonic irradiation apparatus. At least one of the piezoelectric resonator and the battery may desirably be located so that positions at which electrodes are drawn are located at the upper part of the case, that is, above the water surface, when the apparatus is floated on the water surface. Further, a battery part and a vibrator part including a circuit may be divided, and a part of battery and a control unit and a part of the resonator and the acoustic diffusion layer may be divided, and these parts may be mechanically and electrically connectable.

A sound device which generates audible sound of 20 Hz to 2000 Hz is desirably provided in the ultrasonic irradiation apparatus of the present invention. User's favorite music is downloaded and is played during irradiation of the ultrasound, so that a relaxation effect is improved and usage time is known by the music play time. In addition, a portable electronic device can be held easily by this apparatus so that the user can use music, images, and health care information. As conventionally known, the effect of this ultrasonic irradiation apparatus can be improved by implementing in parallel with appropriate muscular training and medication.

Further, the present invention is an ultrasonic irradiation system including the ultrasonic irradiation apparatus, wherein the ultrasonic irradiation apparatus is provided in at least one of a tank capable of receiving human or mammals and filled with water, on a water surface or in the water of the tank, and the ultrasonic irradiation apparatus is provided in the water in the tank capable of radiating ultrasound.

An ultrasound reflector with 80% or greater of ultrasonic reflectance for reflecting and diffusing ultrasound is attached to at least 80% or greater of a surface area of an inner wall surface of the tank. The ultrasound reflector is a composite material, density of a tank inner surface material of a tank outer surface material thereof is 0.01 g/cm³ to 0.1 g/cm³, and is made of a foamed material which is an organic material containing gas. This ultrasound reflector consists of a sheet with easy to attach and remove. The sheet may be a composite material including a waterproof material on a front surface and an organic material containing gas on a back surface. A surface of the sheet of the ultrasound reflector is coated with aluminum which is evaporated film. The surface may be an irregular surface and an organic material containing 90 to 99 volume % of gas may be provided on the back surface. The ultrasound reflector may be multilayer film using a rubber material, PET film, and so forth instead of metallic aluminum. The ultrasonic irradiation system includes a bubble generator which discharges air bubbles of 0.01 mm to 10 mm in diameter into the water, and may use the air bubbles discharged into the water together.

The present invention is an ultrasonic irradiation method in which at least one of the parts of the ultrasonic irradiation apparatus or the ultrasonic resonator is placed in the tank, the ultrasonic resonator is floated and moved on the water, and multi-ultrasound with different frequencies and PRFs are automatically and continuously generated from the ultrasonic irradiation apparatus, the ultrasound is reflected on an inner wall and the water surface of the tank, and stimulation by the low intensity multi-ultrasound is applied to the whole body human or mammals in the water.

A temperature of water in the tank is set to be 37° C. to 42° C., and acoustic wave stimulation is applied to the whole body or a part of the body of human or mammals located in the tank of which height is 0.3 m to 1 m from the bottom surface. The tank is a bathtub, and intensity spatial average temporal average (Isata) of 25 mW/kg to 1000 mW/kg per body weight of a part to which acoustic wave stimulation may be applied to human or mammals in the bathtub. In the ultrasonic irradiation method, the ultrasonic irradiation apparatus continuously or intermittently irradiates human or mammals in the water with the ultrasound 10 to 60 min/day, 2 to 7 times/week, for 2 to 50 weeks. Further, instruction of a training method may be output from a loudspeaker of audible sound of the ultrasonic irradiation apparatus, or a mobile phone. The method of muscular training may be output from a loudspeaker built in the ultrasonic irradiation apparatus.

Regarding intensity of conventional low intensity pulse ultrasound for fracture treatment promotion or a medical ultrasonic diagnostic apparatus, sound intensity is measured on an ultrasound radiation surface of a probe, and expressed by numerical value in the unit of mW/cm² by Isata (intensity of spatial-average temporal-average). Usually, in a LIPUS apparatus for the healing of bone fracture treatment, Isata is 30 mW/cm², and in an ultrasonic treatment instrument using a warm temperature effect, Isata is 1 W/cm² to 3 W/cm². However, if the whole body is irradiated with ultrasound, it is considered to be appropriate to express the total ultrasound power by mW/kg converted per body weight. This is the same idea with the dosage.

The sound intensity in the ultrasonic irradiation system of the present invention is from 25 mW/kg to 1 W/kg of Isata per body weight of a part of human or mammals to which sound wave stimulation is provided. This value is quite small when compared with a conventional fracture treatment ultrasound apparatus. This is because LIPUS stimulation is provided to a large area of about 10,000 cm² of almost the whole body excluding head. However, even if Isata is low, the maximum ultrasound intensity Isptp (Intensity of spatial-peak temporal-peak) of equal to or greater than 100 mW/cm² is momentarily irradiated, and it is considered that this acts on an action potential of various living body cells and exhibits effective effects.

### Effect of the Invention

The ultrasonic irradiation apparatus and the system of the present invention are of simple structure, inexpensive, safe, and small in size. This apparatus can irradiate ultrasonic energy effectively and more uniformly in a tank, such as a bathtub of business use and home use. Further, according to an ultrasonic irradiation method of the present invention, it is also easy to irradiate almost the whole body with LIPUS of various frequencies and PRFs as uniformly as possible. Therefore, blood circulation promotion, pain relief, muscle pain relief, arthritic pain relief, wound healing, lipolysis, losing of weight, promotion of hair fostering, blood pressure lowering, skin activation, eyesight restoration, and so forth which have been reported as effects of bathing and ultrasound stimulation through animal experiments, and so forth are expectable. Further, effects of healing of bone fracture treatment which is an illness relevant to bones, treatment and prevention of knee osteoarthritis, lumbar spinal canal stenosis, and osteoporosis are exhibited. This apparatus especially exhibits effects of health promotion, relaxation, prevention of illness, keeping of athletic ability of elderly athletes, and improvement of quality of life (QOL) of elderly people. In addition, this apparatus exhibits effects of prevention and treatment of fracture, and treatment and recovery of tendons and muscles of racehorse among mammals, for example.

According to the ultrasonic irradiation system and the ultrasonic irradiation method of the present invention, almost the whole body excluding the head, especially the backbone and the femur which have hematopoiesis can be effectively irradiated with ultrasound. It is well known that the blood and the lymph important for the treatment and the prevention of illness are mainly made by the bone marrow in human adults. With the ultrasonic irradiation system and the ultrasonic irradiation method of the present invention, LIPUS stimulation can be provided to the skeleton of the whole body, especially to the bone marrow of the backbone and the femur, so that the living body is activated and vitality is improved easily. In addition, healing of bone fracture treatment and treatment and prevention of knee osteoarthritis, lumbar spinal canal stenosis, and osteoporosis which are well-known illness relevant to bones can be performed. Further, the ultrasonic irradiation system and the ultrasonic irradiation method especially contribute to keeping of athletic ability, rehabilitation, health promotion, prevention of illness, hair restoration, eyesight restoration, QOL improvement of elderly people aged 60 and over, and rehabilitation of racehorses and so forth.

### BRIEF DESCRIPTION OF THE DRAWINGS

FIG. 1 is a schematic diagram of an ultrasonic irradiation apparatus of a first embodiment of the present invention.

FIG. 2A is a graph illustrating time dependency of ultrasound intensity, and FIG. 2B a graph in which a frequency is constant whereas an output level is changed in order to change the ultrasound intensity.

FIG. 3 is a schematic diagram of an ultrasonic irradiation apparatus of a second embodiment of the present invention.

FIGS. 4A and 4B are schematic diagrams of an ultrasonic irradiation apparatus of a third embodiment of the present invention.

FIGS. 5A-5C are schematic diagrams of an ultrasonic irradiation system of a fourth embodiment of the present invention.

FIG. 6 is a schematic diagram of an ultrasonic irradiation system of a fifth embodiment of the present invention.

FIG. 7 is a schematic diagram of an ultrasonic irradiation system of a sixth embodiment of the present invention.

FIGS. 8A and 8B is a schematic diagram illustrating an example of an ultrasonic oscillator of a sixth embodiment of the present invention, and FIG. 8B is a schematic diagram illustrating another example.

FIG. 9 is a schematic diagram of an ultrasonic irradiation system of a seventh embodiment of the present invention.

FIG. 10 is an X-ray photograph on Jan. 7, 2014 of a knee of a patient of knee osteoarthritis, in which example a meniscus of a right knee is worn out.

FIG. 11 is an X-ray photograph on Feb. 25, 2017 of the knee of the patient illustrated in FIG. 10 undergone acoustic stimulation for two years with an ultrasonic irradiation apparatus of the present invention, in which example the meniscus of the right knee has recovered.

### MODE FOR CARRYING OUT THE INVENTION

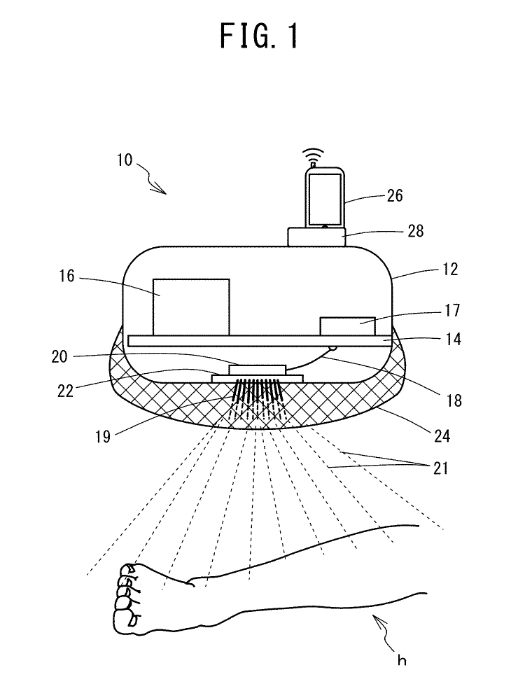

Hereinafter, an embodiment of the present invention will be described with reference to the drawings. FIG. 1 illustrates a first embodiment of the present invention. an ultrasonic irradiation apparatus 10 of this embodiment illustrates a basic configuration which includes a waterproof case 12, a substrate 14 inside the case 12, and a driving unit consisting of a power supply 16, such as a battery, and a control unit 17 including a control circuit, and so forth on an upper surface of the substrate 14. A lead wire 18 is connected to an unillustrated terminal of the substrate 14 and is connected to an unillustrated terminal of an ultrasonic resonator 20 which is a piezoelectric resonator provided below the substrate 14 on an underside of the substrate 14.

The ultrasonic resonator 20 is attached to the case 12 via an acoustic matching layer 22 and an unillustrated protection film, so that high intensity ultrasound (HIUS) 19 can be radiated from a surface of the case 12. A thickness of the acoustic matching layer 22 is multiples of ¼ of an underwater wavelength λ of the used ultrasound, and a contour shape of the acoustic matching layer 22 is 120% to 200% of the ultrasonic resonator 20 in magnitude in a project area. The ultrasonic resonator 20 and an extraction position of the lead wire 18 of the power supply 16, which is a battery, desirably are positioned at an upper part of the case 12 as much as possible. This suppresses damage to the power supply 16 or the control unit 17 to the minimum, and enables replacement of a damaged portion if the case 12 is flooded for a certain reason. The acoustic matching layer 22 may have at least two types of materials. A thickness of the acoustic matching layer 22 may be multiples of ¼ of an underwater wavelength λ of the used ultrasound, and a contour shape of the acoustic matching layer 22 may be 120% to 200% of the ultrasonic resonator 20 in a project area.

An acoustic diffusion layer 24 made of an ultrasound diffusing material is provided outside of the case 12 at a part at which the ultrasonic resonator 20 is attached. There is a large difference between an acoustic impedance of the acoustic diffusion layer 24 and an acoustic impedance of water, and the ultrasound diffusing material of the acoustic diffusion layer 24 desirably is a metal member, such as a wire net, with an acoustic impedance of 40 or greater. The acoustic diffusion layer 24 made of an ultrasound diffusing material controls high intensity ultrasound 19 into low intensity ultrasound (LIUS) 21 with relatively low intensity per unit area, diffused widely, and irradiated in a wide area. The ultrasound diffusing material is made of a porous metal wire net of which opening is λ-λ/10 with respect to the underwater wavelength λ of the used ultrasound. A fixable support base 28 which removably holds a portable electronic device 26, such as a smart phone, as audio equipment is provided in an upper part of the case 12.

The ultrasonic resonator 20 is a piezoelectric element which oscillates ultrasound when a voltage is added thereto, and uses thickness vibration and radial vibration. A resonance frequency of the ultrasound to oscillate is equal to or greater than 0.3 MHz and equal to or less than 5 MHz. However, the ultrasonic resonator 20 generates not only the frequency component of a fundamental wave but its harmonics, and these are also used effectively. As the piezoelectric element of the ultrasonic resonator 20, a PZT based ceramic resonator which has a large electromechanical coupling coefficient and is obtained inexpensively is mainly selected. However, since the PZT resonator contains 50% or greater of lead oxide which affects an environment, it is necessity to collect and perform suitable process if the apparatus breaks. Therefore, employing a lead-free piezoelectric material consisting mainly of alkaline niobate compound is desirable. The ultrasonic resonator 20 may include an unillustrated acoustic backing layer usually used in a probe of medical diagnostic imaging equipment on a surface on an opposite side of a radiation direction.

As usage of the ultrasonic irradiation apparatus 10 of the present embodiment, as illustrated in FIG. 1, the acoustic diffusion layer 24 is placed to face a part of a human body h so that the low intensity ultrasound 21 is applied to the part to be treated of the human body h. Further, as described later, the ultrasonic irradiation apparatus 10 is floated on a bathtub so as to provide low intensity ultrasound stimulation to the whole body excluding the head.

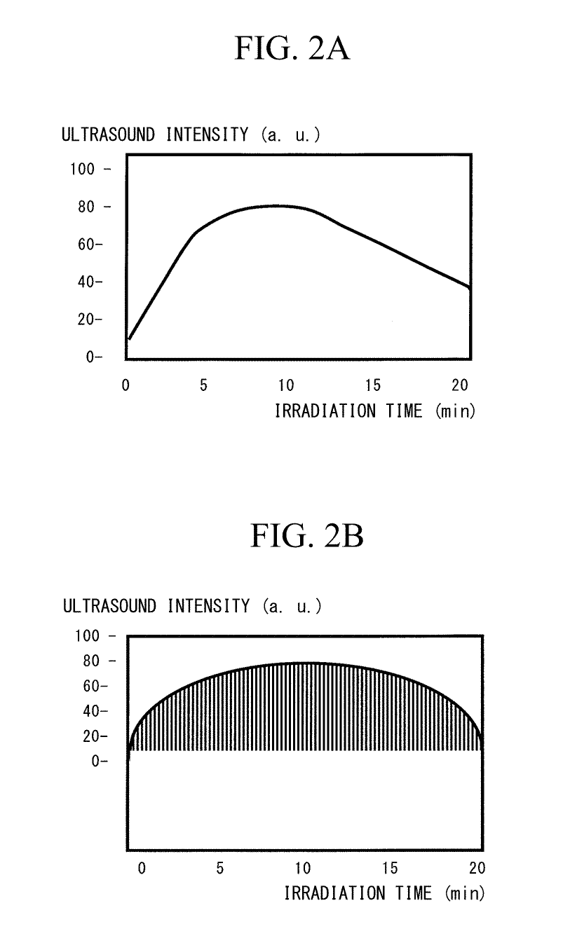

The ultrasound intensity of the ultrasonic irradiation apparatus 10 when in use can be set suitably. For example, as illustrated in FIG. 2A, after gradually increasing the intensity with time, the intensity may be gradually lowered. Alternatively, as illustrated in FIG. 2B, oscillation may be made in a pulse form to gradually increase intensity of the ultrasound, and then the intensity is gradually lowered similarly in the irradiation. A change in the ultrasound intensity may be made by changing the PRF of the used ultrasonic pulse or changing the amplitude with time. Especially an effect of increasing bone growth using a principle of crystal growth is exhibited by changing the oscillated ultrasound as illustrated in FIG. 2B, in which a frequency is constant whereas an output level is changed.

In order to control an irradiation area of the low intensity ultrasound 21 by the ultrasonic irradiation apparatus 10, a concave or convex acoustic lens and so forth used for an ultrasound probe of a medical ultrasonic diagnostic apparatus may desirably be used. The ultrasound may be a continuous wave, but more desirably a pulse wave generated intermittently. As the pulse wave, ultrasound of which pulse period is 0.001 to 2 seconds and duty factor is 10% to 60% is used, for example. Various types of waveforms, such as a sine wave, a square wave, and a triangular wave, may be used for the ultrasound. However, a desirable pulse repetition frequency (PRF) is 0.5 Hz to 2 Hz, and a desirable duty factor is 20% to 50% which is close to those of the heart pulses. An especially desirable PRF is a combination of about 1 Hz of 1 s close to a heart rate to about 500 Hz (a period of several ms) which is a transmission speed of a nervous system of human or mammals. Thus, various living body cells which are diversity can further be activated in shorter time by using various PRFs and various duty factors.

Sound intensity Isata of the low intensity ultrasound 21 may desirably be 25 mW/kg to 1 W/kg per body weight of the part to be irradiated with the ultrasound. If Isata is equal to or smaller than 25 mW/kg, the effect to the growth and repair related to the bones or the skin is very small even after 30 weeks or more elapse. If Isata is equal to or greater than 1 W/kg, prolonged exposure may be harmful to human or mammals, and the apparatus may increase in size. The sound intensity of the ultrasonic irradiation apparatus 10 is preferably 100 mW/kg to 300 mW/kg.

The ultrasonic irradiation apparatus 10 may further include a sound wave device, such as a loudspeaker, which generates audible sound of 20 Hz to 2000 Hz. The audible sound may preferably be music, and outputting favorite music of a user may provide a relaxation effect and a timer effect for indicating operating time. The ultrasonic irradiation apparatus 10 may have additional functions, such as an elapsed time indicator, blinking indicator of an operating state, warning sound, a communication function, radio, TV, video, user safety check, and alarming.

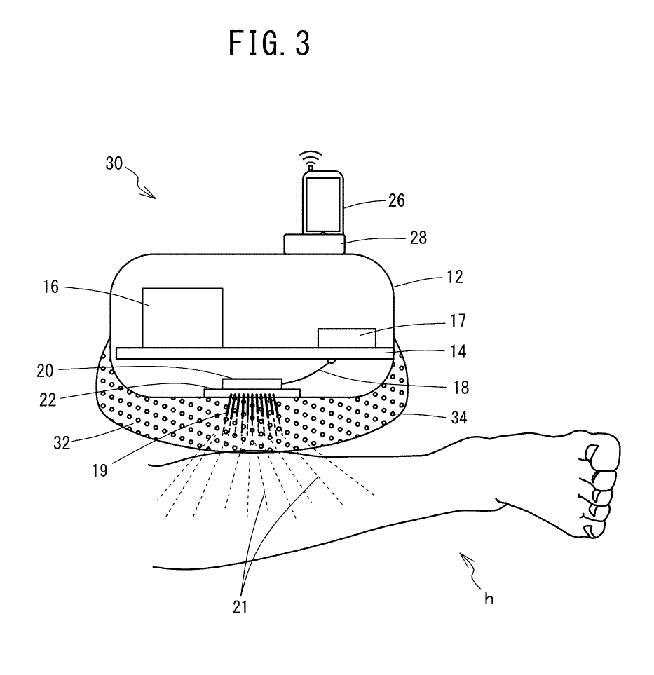

Next, an ultrasonic irradiation apparatus 30 of a second embodiment of the present invention will be described with reference to FIG. 3. The same components as those in the above embodiment are denoted by the same reference numerals, and description thereof will be omitted. The ultrasonic irradiation apparatus 30 of the present embodiment is the same in configuration as that illustrated in FIG. 1, and different in an ultrasound diffusing material of the acoustic diffusion layer.

An acoustic diffusion layer 32 of the ultrasonic irradiation apparatus 30 of the present embodiment is made of a foamed resin material containing 90 to 99 volume % or more of gas, such as air, and air bubbles and contains foam 34. The acoustic diffusion layer 32 comes into direct contact with a human body to irradiate the human body with low intensity ultrasound 21. The foamed resin desirably is polystyrene and polyurethane. The ultrasound diffusing material used here contains equal to or greater than 90 volume % of gas because, if the gas content is equal to or less than 90 volume %, density of the ultrasound diffusing material increases and effective scattering of high intensity ultrasound 19 becomes difficult. If the gas content is equal to or greater than 99 volume %, mechanical strength of the ultrasound diffusing material is lowered and operability is reduced. Polystyrene foam containing 95 to 98 volume % gas is especially preferably used as an ultrasound diffusing material.

The high intensity ultrasound 19 oscillated by the ultrasonic resonator 20 is irregularly reflected on the foam 34 including air and is changed into the low intensity ultrasound 21 with relatively lower intensity, is diffused in a wider radiation direction, and reaches the human body h. An ultrasound gel generally and widely used for a probe for ultrasonic diagnoses, and so forth may desirably be used between the human body h and the ultrasonic irradiation apparatus 30 to improve contactability.

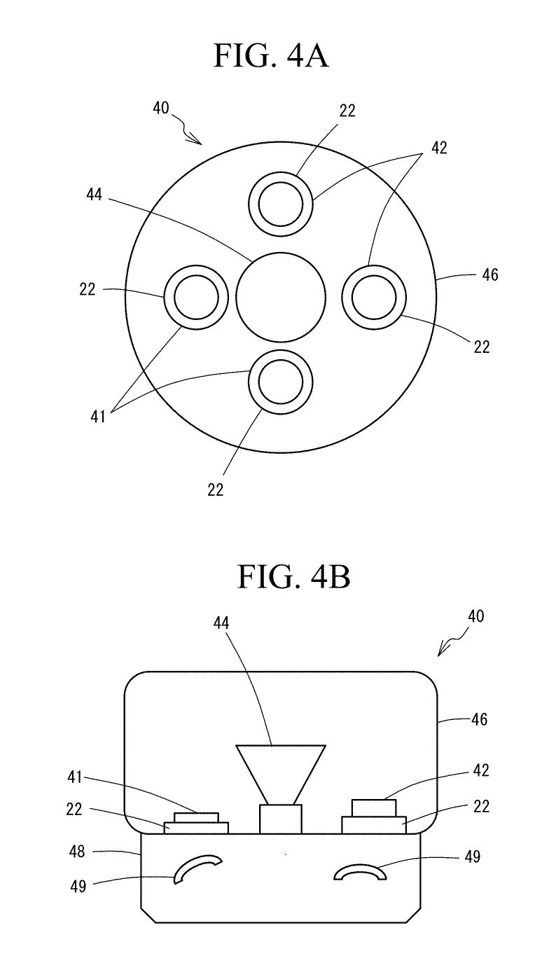

Next, an ultrasonic irradiation apparatus 40 of a third embodiment of the present invention will be described with reference to FIGS. 4A and 4B. The ultrasonic irradiation apparatus 40 of the embodiment illustrated in FIGS. 4A and 4B includes two ultrasonic resonators 41 and two ultrasonic resonators 42 of two different frequencies, and is provided with a loudspeaker 44 which outputs music of audible sound, and so forth. An acoustic diffusion layer 48 holding an ultrasonic diffusion member 49 made of an ultrasound diffusing material is provided on an outer surface of a case 46 of this apparatus. The acoustic diffusion layer 48 holding the ultrasonic diffusion member 49 is made of various foamed resin materials containing 90% or greater of air or other gas. Two pairs of ultrasonic diffusion members 49 each corresponding to the ultrasonic resonators 41 and the ultrasonic resonators 42 are provided inside of the acoustic diffusion layer 48. The two pairs of the ultrasonic diffusion members 49 are set as mutually different suitable orientations. The acoustic diffusion layer 48 may be made of a metal member, such as a wire net.

The acoustic diffusion layer 48 can scatter and diffuse highly directive high intensity ultrasound 19 in a controlled manner by changing the material, the shape, and the number of holes, and the position, and can irradiate human or mammals with lowered low intensity ultrasound 21 in a wide area. Further, by changing the orientation and the position of each of the ultrasonic diffusion members 49, highly directive high intensity ultrasound 19 is diffused to provide low intensity ultrasound stimulation to the entire part of a human and mammals to which sound wave stimulation is provided in a wide area. Desirably, the two ultrasonic resonators 41 and the two ultrasonic resonators 42 continuously and automatically generate two frequencies by an unillustrated driving unit. Alternatively, ultrasound of at least two different frequencies may be generated by a single piezoelectric resonator.

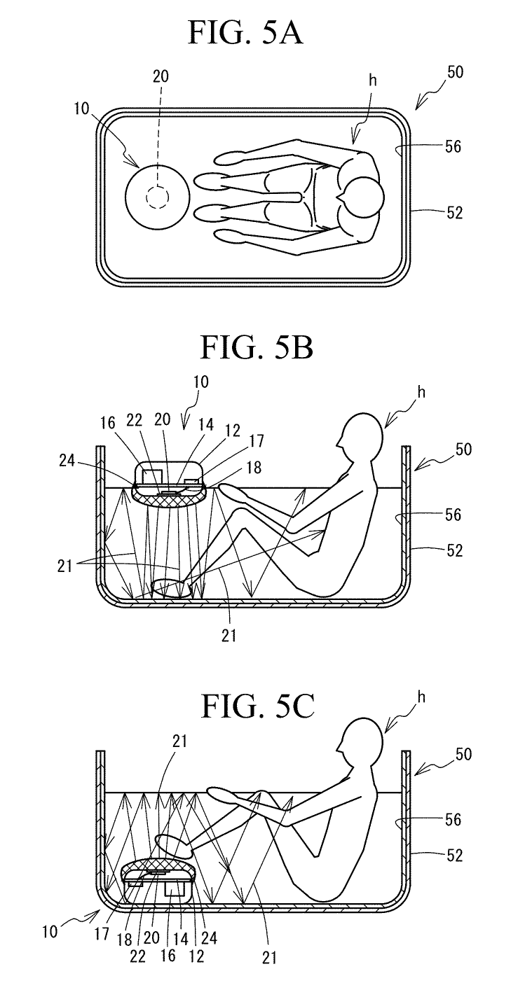

Next, an ultrasonic irradiation apparatus and an ultrasonic irradiation system 50 of a fourth embodiment of the present invention will be described with reference to FIGS. 5A-5C. The same components as those in the above embodiments are denoted by the same reference numerals, and description thereof will be omitted. FIGS. 5A-5C illustrate a system in which the ultrasonic irradiation apparatus 10 of the above-described first embodiment is floated or fixed in a tank 52, such as a bathtub, and provides whole body ultrasound stimulation to the human body h.

It is desirable that the center of gravity of the floating ultrasonic irradiation apparatus 10 is adjusted off-balance so that an ultrasound radiation surface of an ultrasonic resonator 20 is inclined at 3° to 30° with respect to a water surface. Further, in order to control an irradiation area of low intensity ultrasound 21 by the ultrasonic irradiation apparatus 10, an acoustic lens and so forth used for an ultrasound probe of a medical ultrasonic diagnostic apparatus as described above may be used.

The ultrasound may be a continuous wave, but more desirably a pulse wave generated intermittently. As the pulse wave, ultrasound of which period is 0.001 to 2 seconds and duty factor is 5% to 60% is used, for example. Various types of waveforms, such as a sine wave, a square wave, and a triangular wave, may be used for the ultrasound. A desirable pulse repetition frequency (PRF) is 0.5 Hz to 2 Hz, and a desirable duty factor is 20% to 50% which is close to those of the heart pulses. An especially desirable PRF is a combination of from about 1 Hz of which period is 1 s close to a heart rate to about 500 Hz of which period is several ms which is a transmission speed of a nervous system of a human body, and 20 ms to 40 ms (50 Hz to 25 Hz) which is an actual action potential of living body cells.

Sound intensity Isata of the low intensity ultrasound 21 may desirably be 25 mW/kg to 1 W/kg per body weight of the part to be irradiated with the ultrasound. If Isata is equal to or smaller than 25 mW/kg, the effect to the growth and healing related to the bones or the skin is very small even after 30 weeks or more elapse. If Isata is equal to or greater than 1 W/kg, prolonged exposure may be harmful to a human body, and the apparatus may increase in size. The sound intensity of the ultrasonic irradiation apparatus 10 is preferably 100 mW/kg to 300 mW/kg.

The ultrasonic irradiation apparatus 10 may further include a sound wave device which generates audible sound of 20 Hz to 2000 Hz. The audible sound may preferably be music, and outputting favorite music of a user may provide a relaxation effect and a timer effect for indicating operating time. The ultrasonic irradiation apparatus 10 may have additional functions, such as an elapsed time indicator, blinking indicator of an operating state, warning sound, a communication function, radio, TV, video, user safety check, and alarming.

As usage of the irradiation system 50 of the present embodiment, as illustrated in FIGS. 5A-5C, the ultrasonic irradiation apparatus 10 is floated on or tied under water 54 in the tank 52, such as a bathtub, the high intensity ultrasound 19 is radiated from the ultrasonic resonator 20 toward the water 54, so that the human body h is irradiated with the low intensity ultrasound 21 as uniform as possible, while effectively using irregular reflection of the ultrasound on a bottom surface and a side surface of the tank 52.

An ultrasound reflector 56 with density of 0.1 to 0.01 and containing gas is desirably attached to an area of 80% or greater of the side surface and the bottom surface which is an inner surface of the tank 52. The ultrasound reflector 56 is made of foamed resin containing 90 to 99 volume % of gas. By using such an ultrasound reflector 56, equal to or greater than 90% of the low intensity ultrasound 21 can be effectively reflected and effectively applied to the human body h.

In the present embodiment, the low intensity ultrasound stimulation is provided to the human body h in the tank 52 of which height from a bottom surface to the water surface of the water 54 is 30 cm to 1.0 m. If the height to the water surface is equal to or less than 30 cm, the depth is insufficient even for a small elderly person to sink the entire human body h below the water surface, and if the height is 1.0 m or greater, there is a possibility of drowning for elderly people. The optimal depth is 35 cm to 50 cm at which the elderly people can easily breath at a seating position. The frequency of the used ultrasound can be selected from 0.3 MHz to 5 MHz. Frequencies of 0.3 MHz to 2 MHz are suitable to provide ultrasound power to bones located at a deep part more than 10 cm from the body surface and, 2 MHz to 5 MHz are suitable to stimulate bones, muscles, and joints located at a position from 3 cm or closer to the body surface. More desirably, these frequencies are used in combination and in series to stimulate various bones at different parts of the whole body simultaneously. If the frequency of the used ultrasonic resonator is equal to or greater than 5 MHz, attenuation under the water containing a large amount of air, in the skin, fat, and muscles becomes greater and obtaining necessary ultrasound intensity becomes difficult. The tank 52 may desirably have a side surface made of a material having an acoustic impedance of equal to or greater than 3 MRayls and equal to or less than 50 MRayls. As the material, FRP resin, concrete, marble, agate, jade, crystal, glass, metal, and so forth may be used. Mental effects can be provided by using agate, jade, crystal, and so forth which are recognized especially as powerful stones. When the present embodiment is applied to mammals, the size of the tank 52 may be changed in accordance with the magnitude of the animals.

The human body h may be irradiated with the sound wave at any part except for the head under the water, but especially desirably irradiated with the leg and the back. This is because the femur and the backbone have large spines and thus have especially important hematopoiesis.

The ultrasonic irradiation apparatus 10 generates ultrasound 10 to 60 min/day, 2 to 7 times/week, for 2 to 50 weeks, for example. In the short time of 10 minutes or shorter, an ultrasound irradiation effect, such as a health care effect, is small, and even if irradiation is continued in the same tank 52 for 60 minutes or longer, the effect does not greatly differ and side effects, such as fatigue, may be caused. An irradiation frequency is 2 to 7 times/week, and more preferably 3 to 5 times/week. Although the irradiation time of about 20 weeks is effective, 30 weeks or longer is more preferable. Regarding the water temperature in the tank 52, 37° C. to 42° C. which are higher by 2 to 5° C. than the body temperature are desirable to encourage perspiration and to promote blood flow. If the water temperature is at or below the body temperature, the effect is not exhibited, and if the water temperature exceeds 42° C., especially elderly people feel severe fatigue when bathed for 20 minutes or longer. The optimal temperature is 39° C. to 41° C., in which temperature range bone formation can be promoted. The same conditions can be applied to mammals.

The ultrasonic irradiation apparatus 10 floated on the water moves to the front, back, left, and right or inclines to different directions with the move of the water surface in the tank 52, and the position and the angle of the ultrasonic resonator 20 change continuously, so as to irradiate the human body h and the mammals with the low intensity ultrasound 21 further uniformly by an effect of the acoustic diffusion layer 24.

According to the ultrasonic irradiation apparatus 10, the ultrasonic irradiation system 50, and the ultrasonic irradiation method of the present embodiment, the ultrasound stimulation is uniformly provided to the human body h excluding the head by the ultrasonic irradiation apparatus 10 of a simple structure, while freely changing the radiation direction of the low intensity ultrasound 21. Further, the low intensity ultrasound stimulation can be uniformly provided to the human body h in the tank using irregular reflection of the ultrasound from the bottom surface and the side surface of the tank 52. The ultrasonic irradiation apparatus 10 is portable and can be used in a large-sized tank other than a bathtub. The same effect is exhibited if the number of the ultrasonic irradiation apparatuses 10 is increased, and the ultrasonic irradiation apparatus 10 is temporarily stationary and fixed to the tank 52. By using these apparatuses, the low intensity ultrasound stimulation can be provided to many patients simultaneously and still more uniformly. The ultrasonic irradiation apparatus 10 can be small-sized (0.2 kg to 2 kg in weight) and is easily carried by elderly people.

When the apparatus is not in use, the apparatus can be removed from the tank and can be charged and cleaned easily, so that the apparatus is easily kept clean. Therefore, the apparatus is suitable for mass production, and can be manufactured with significantly reduced production cost due to small maintenance management cost for maintenance and collection. The same effect can be exhibited also for mammals.

Although the ultrasonic irradiation apparatus 10, the ultrasonic irradiation system 50, and the method can be used for almost all ages regardless of gender, these especially substantially contribute to treatment and prevention of knee osteoarthritis and osteoporosis of elderly people aged 60 and over.

The ultrasonic irradiation system 50 and the ultrasonic irradiation method of the present embodiment may use the ultrasonic irradiation apparatus 40 provided with the two or more ultrasonic resonators 41 and the two or more ultrasonic resonators 42 illustrated in FIGS. 4A and 4B. By using the ultrasonic irradiation apparatus 40, it is possible to radiate the low intensity ultrasound 21 more widely in more than two different directions with a single ultrasonic irradiation apparatus, radiate the low intensity ultrasound 21 uniformly in the tank 52, and reduce the number of necessary ultrasonic irradiation apparatuses. A plurality of ultrasonic resonators 41 and 42 may have different frequencies, PRFs, duty factors, and sound intensities, which can be set suitably.

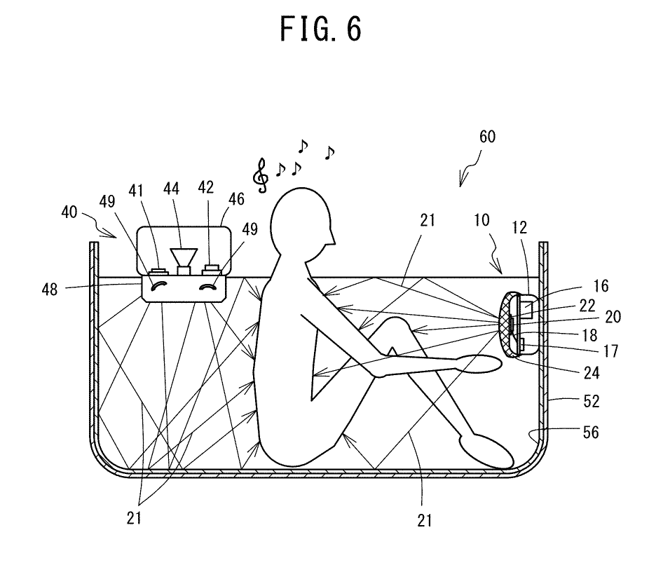

Next, an ultrasonic irradiation apparatus and an ultrasonic irradiation system 60 of a fifth embodiment of the present invention will be described with reference to FIG. 6. The same components as those in the above embodiments are denoted by the same reference numerals, and description thereof will be omitted. In the ultrasonic irradiation system 60 of the present embodiment, the ultrasonic irradiation apparatus 40 is floated on a water surface of a tank 52, such as a bathtub, and an ultrasonic irradiation apparatus 10 is detachably attached to an inner wall of the tank 52 in the water. As the ultrasonic irradiation apparatus, an apparatus similar to the ultrasonic irradiation apparatuses 10, 30, and 40 of the first to the third embodiments is suitably used.

In the ultrasonic irradiation apparatuses 10, 30, and 40 used in the present embodiment, high intensity ultrasound 19 radiated by each ultrasonic resonator 20 and passed through an acoustic matching layer and a protective layer (not illustrated) is attenuated and diffused by the acoustic diffusion layer s 24, 32, and 48 disposed on the front side in the radiation direction and made of an ultrasound diffusing material and then radiated. The attenuated and diffused low intensity ultrasound 21 repeats irregular reflection on an interface between the inner wall and an outer wall of the tank 52 and the water surface, and is eventually radiated uniformly onto the human body h. The low intensity ultrasound 21 passes through the skin, fat, and muscles which are tissues inside of the human body, and most of the low intensity ultrasound 21 reaches the bones which are hard tissues and are attenuated inside the bones and converted into thermal energy. Then the low intensity ultrasound 21 provides stimulation to the bones, increases bone temperature, and contributes to an increase in osteoblast, and so forth. The user can receive operation instructions about necessary movement, for example, from the loudspeaker 44 which outputs audible sound, such as music. Therefore, the apparatus can be used in various ways.

According to the ultrasonic irradiation system 60 and the ultrasonic irradiation method of the present embodiment, the ultrasound stimulation can be uniformly provided to the human body h excluding the head by selecting a plurality of ultrasonic irradiation apparatuses 10, 30, and 40 and freely changing the radiation direction of the low intensity ultrasound 21. Further, the ultrasound stimulation can be uniformly provided to the human body h in the tank using the irregular reflection of the ultrasound from the bottom surface and the side surface of the tank 52.

The ultrasonic irradiation apparatuses 10, 30, and 40 are portable and may be used in large-sized tanks other than a bathtub. The user can receive operation instructions about necessary movement, for example, from the loudspeaker 44 which outputs audible sound, such as music. Therefore, the apparatus can be used in various ways. Further, the same effect is exhibited if the number of the ultrasonic irradiation apparatuses 10 and the like is increased, and the ultrasonic irradiation apparatus 10 and the like is temporarily stationary and fixed to the tank 52 and a wall of the tank. By using these apparatuses, the low intensity ultrasound stimulation can be provided to many patients simultaneously and still more uniformly. The ultrasonic irradiation apparatuses 10, 30, and 40 can be small-sized (0.2 kg to 2 kg in weight) and is easily carried by elderly people. When the apparatuses are not in use, the apparatuses can be removed from the tank and can be charged easily, so that the apparatuses can be easily kept clean. Therefore, the apparatus is suitable for mass production, and can be manufactured with significantly reduced production cost due to small maintenance management cost for maintenance and collection.

Each of the ultrasonic irradiation apparatuses 10, 30, and 40 may be floated on the water surface in a movable manner, or may be temporarily fixed. The ultrasound diffusing material of the acoustic diffusion layer s 24, 32, and 48 are made of metal, or foamed resin containing 90% or greater of air, gas, such as air, having a large difference in an acoustic impedance with water. Further, the ultrasound reflector 56 used for the inner wall of the tank 52 may desirably be metal or foamed resin which includes 90% or greater of gas, such as air, having a large difference in acoustic impedance with water, and may be FRP usually used for a bathtub material having a foamed material attached to a back surface thereof. These structures and conditions are the same when used for mammals.

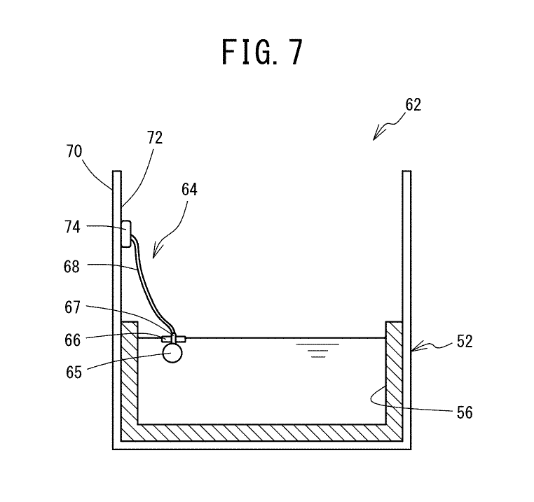

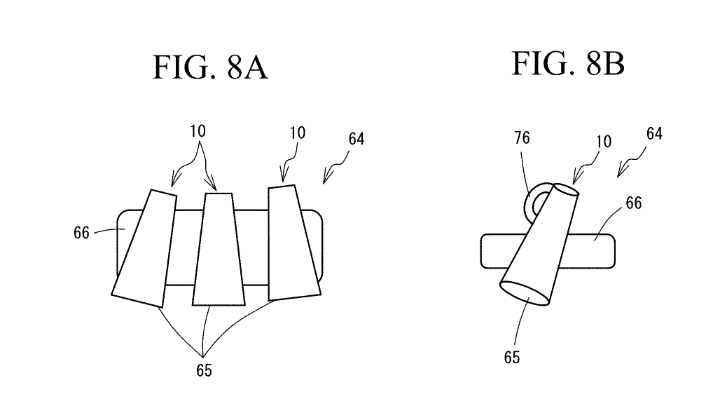

Next, an ultrasonic irradiation apparatus and an ultrasonic irradiation system 62 of a sixth embodiment of the present invention will be described with reference to FIGS. 7 and 8. The same components as those in the above embodiment are denoted by the same reference numerals, and description thereof will be omitted. In the ultrasonic irradiation system 62 of the present embodiment, an ultrasonic oscillator 65 which includes a piezoelectric resonator and an acoustic diffusion layer of an ultrasonic irradiation apparatus 64 is attached to the float 66 and floating on a water surface in a tank 52, such as a bathtub. The ultrasonic oscillator 65 is fixed to the float 66 which is a floatable holding member via a fixing part 67. The ultrasonic oscillator 65 which includes the piezoelectric resonator is connected to a driving unit 74 fixed to an inner wall 72 of a bathroom 70 via a connection line 68. A power supply which is a battery and a control unit are provided in the driving unit 74, so that the ultrasonic oscillator 65 is supplied with power provided with drive control. Therefore, a degree of freedom of water-tightness of the power supply 16 and the control unit 17, and magnitude of the power supply 16 and so forth is increased, so that the apparatus becomes easy to use.

Regarding the ultrasonic oscillator 65 of the ultrasonic irradiation apparatus 64, as illustrated in FIG. 8A, a plurality of ultrasonic oscillators 65 may be fixed to a single float 66. A plurality of ultrasonic oscillators 65 may have different radiation directions of the ultrasound, different frequencies of the ultrasound, and different PRFs. This enables more effective radiation of the ultrasound in a wide area. As illustrated in FIG. 8B, a single ultrasonic oscillator 65 may be fixed to a single float 66, and a handle 76 may be provided in the ultrasonic oscillator 65. Since the handle 76 is provided, handleability is improved. Further, the handle 76 and an unillustrated lid may desirably be provided to effectively radiate heat which is generated inside of the apparatus. Therefore, if overheating occurs inside the apparatus, the heat is radiated effectively and the apparatus is cooled, and internal electronic components can be maintained desirably.

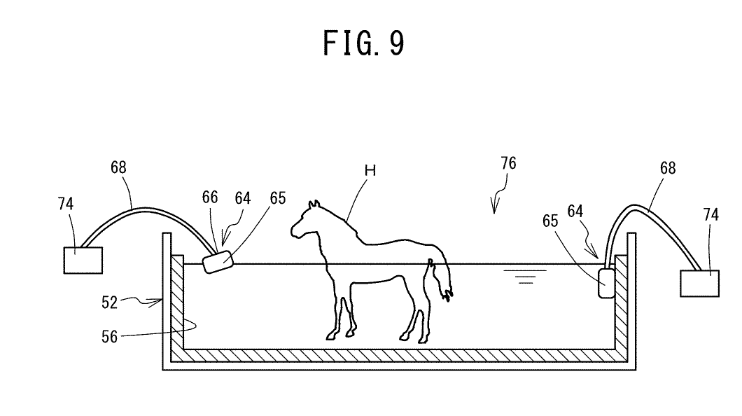

Next, an example in which an ultrasonic irradiation apparatus 64 and an ultrasonic irradiation system 76 of a seventh embodiment of the present invention are used to a horse H which is a mammal will be described with reference to FIG. 9. The same components as those in the above embodiments are denoted by the same reference numerals, and description thereof will be omitted. In the ultrasonic irradiation system 76 of the present embodiment, an ultrasonic oscillator 65 which includes a piezoelectric resonator and an acoustic diffusion layer of the ultrasonic irradiation apparatus 64 is attached to a float 66 and floating on a water surface in a tank 52, such as a pool, which can receive a horse H. The ultrasonic oscillator 65 may be located under the water. The ultrasonic oscillator 65 which includes the piezoelectric resonator and the acoustic diffusion layer is connected to a driving unit 74 located outside of the tank 52 via a connection line 68. A power supply and a control unit are provided in the driving unit 74, so that the ultrasonic oscillator 65 is supplied with power provided with drive control. Therefore, a degree of freedom of waterproof of the power supply and the control unit, and magnitude of the power supply and so forth is increased, so that the apparatus becomes easy to use.

The ultrasonic irradiation system 76 of the present embodiment contributes to treatment and prevention of fractures, and treatment and prevention of tendons and muscles of racehorses by applying to mammals, such as racehorses.

The ultrasonic irradiation apparatus, the ultrasonic irradiation system, and the ultrasonic irradiation method of the present invention are not limited to the above embodiments, but may be modified suitably. For example, the ultrasonic irradiation apparatus may be provided in a waterproof cylindrical case made of resin, and positions at which lead wires of the piezoelectric resonator and a power supply thereof are drawn desirably are located inside of the case and at an upper part of the case. This prevents short circuits in the battery of the power supply and the control unit and suppresses damage to the power supply or the control unit is to the minimum, and enables replacement of a damaged portion if the case is flooded for a certain reason. Further, a water leakage sensor may be provided at a lower part of the case to inform the user of abnormality by flashing a lamp upon short circuits.

At least two ultrasonic resonators of the ultrasonic irradiation apparatus may desirably be placed on a side surface of the case which is shaped as a pyramid, a cone, or a sphere, and ultrasound radiation surfaces of the ultrasonic resonators may desirably cross in an angle range of 60° to 200°. This enables wide and efficient radiation on a body surface of a user.

The ultrasonic irradiation apparatus may generate ultrasound while temporarily moving or being fixed at an arbitrary depth under the water other than floating on a water surface. The structure and the material of the case of the ultrasonic irradiation apparatus may be freely selectable from those which reliably hold the ultrasonic resonator and are portable. Further, the size and the shape of the used tank may be changed. The number of the ultrasonic resonators of the ultrasonic irradiation apparatus and the number of the ultrasonic resonators to be placed in the tank may be adjusted in accordance with the size and the shape of the tank so that suitable ultrasound intensity is obtained for each human body. These structures and conditions are the same when the apparatus is used for mammals.

As usage of the ultrasonic irradiation apparatus, the apparatus may come into direct contact with a human body using generally used gel for ultrasound propagation, without using a tank. The apparatus may be used as a conventionally known cosmetic machine and an ultrasonic treatment instrument, or may be used as a massaging machine.

The acoustic diffusion layer of the present invention is not limited to a foaming resin plate or perforated metal plate or a wire net. The same effect of widely diffusing and scattering an ultrasonic beam is exhibited by shapes of a propeller, a windmill, and an umbrella, and a structure in which slit holes are formed in a metal material.

The present invention is one of physical therapies, and the effect is exhibited when used together with a conventionally used ultrasound bath using microbubbles and bubbles or in a bathtub which provides electrical stimulation. Further, the present invention may be implemented in parallel with muscular training performed in rehabilitation. Further, the present invention may be implemented in parallel with a treatment of osteoporosis using medication, and intraarticular injection of polymers hyaluronic acid and plasma which are currently performed widely in orthopedics.

In the present invention, a part of the acoustic matching layer is a transparent organic substance constituting the case in order to easily display notification to the user inside of the case. Transparent silicone rubber, polyurethane rubber, polyethylene terephthalate resin, and polycarbonate resin are especially suitable for this purpose. For elderly people, usage and notification may be displayed with large characters and signs. Motivation to continuously use the ultrasound stimulation apparatus can be improved by placing user's favorite family photo, text, and design in the transparent case.

The acoustic diffusion layer may be formed in a shape of a plant or an animal, and a part thereof may emit light from an LED, for example, while generating ultrasound by the ultrasonic resonator. Further, a training method, a confirmation signal of a physical condition, and so forth may be output from a loudspeaker of audible sound or a portable electronic device provided in the ultrasonic irradiation apparatus. The apparatus, the method, and the system may be used for mammals having the same skeletal structure as that of human, such as dogs, cats, cows, monkeys, and horses, especially for the treatment and damage prevention of tendons, muscles, and bones of racehorses.

### EXAMPLE 1

Next, Examples of the ultrasonic irradiation apparatus, the ultrasonic irradiation system, and the ultrasonic irradiation method of the present invention will be described below. First, as Example 1, an example in which the ultrasonic irradiation system and the ultrasonic irradiation method of the present invention are applied to a 64-years-old male patient with moderate knee osteoarthritis whose body weight excluding the head is 54 kg will be described.

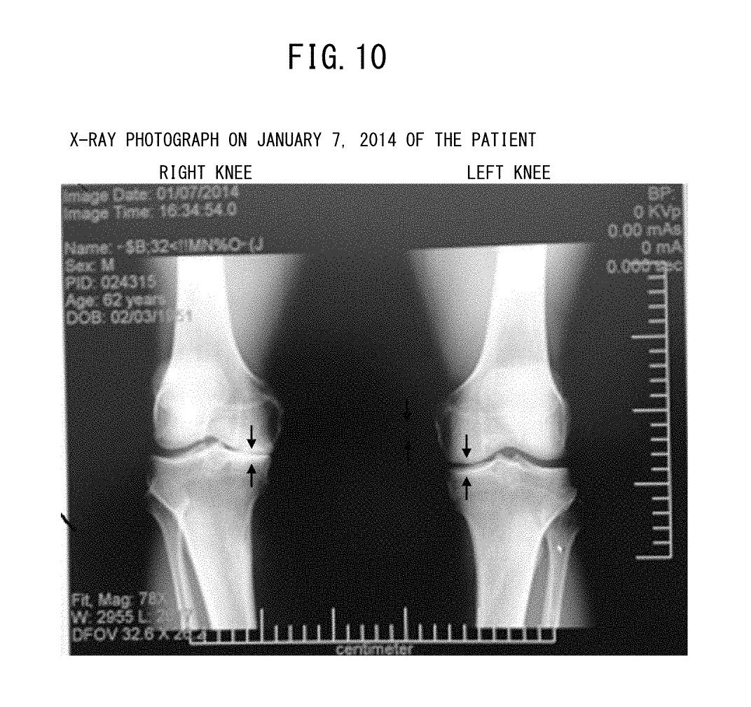

The patient is an elderly amateur soccer player whose about 45-years athlete carrier caused severe burden on his knee and caused a meniscus injury in his right knee. FIG. 10 is an X-ray photograph of both knees of this patient on January, 2014. Especially the meniscus in the right knee is worn and caused a severe pain. At the age of 64.0, 50-m running speed of this patient is 16 seconds due to knee pain.

An average flight distance of 5 times of a soccer ball by instep kick is 25 m by the right foot and 12 m by the left foot. An absolute value of knee extension muscular power (Nm) is 125 Nm before investigation.

As treatment, three ultrasonic irradiation apparatuses 10 and 30 illustrated in FIGS. 1 and 3 are used in a stainless steel home bathtub, sound wave irradiation is applied 20 min/times, 4 or 5 times/week, for 100 weeks. Frequencies of the three ultrasonic irradiation apparatuses 10 and 30 are 0.33 MHz, 0.8 MHz, and 1.5 MHz, respectively. The 0.33 MHz- and 0.8 MHz-ultrasonic irradiation apparatuses 30 use a PZT based ultrasound piezoelectric resonator of which diameter is 25 mm (area is 5 cm²) and Isata is 1,200 mW/cm². The 1.5-MHz ultrasonic irradiation apparatus 10 uses a PZT based ultrasound piezoelectric resonator of which diameter is 25 mm (area is 5 cm²) and Isata is 1,000 mW/cm². All the PZT resonators are the C-203 hard PZT material manufactured by Fuji Ceramics Corporation.

In the 0.33-MHz ultrasonic irradiation apparatus 30 of which period is 3.3 μs, the PRF is 500 Hz, and the duty factor is 40%. Isata is 111 mW/kg per body weight. The acoustic diffusion layer 32 is a 2 mm thick umbrella-shaped polystyrene foam plate having two 1 mm diameter (22% of underwater wavelength λ) holes formed at a central portion. The polystyrene foam plate is placed at a central portion of 2 cm thick silicone rubber and the maximum intensity of the ultrasound in the tank is measured. The result is 30 mW/cm². The acoustic diffusion layer 32 reduces the maximum value of ultrasound intensity to about 2.5% as compared with a case where no acoustic diffusion layer 32 is attached, and an ultrasonic beam can be diffused in a large area.

In the 0.5-MHz ultrasonic irradiation apparatus 30 of which is 2 μs,the PRF is 1 Hz, and the duty factor is 50% at the start time and is changed stepwise into 25% at the completion. Isata is from 111 mW/kg to 56 mW/kg per body weight. The acoustic diffusion layer 32 is a 2-mm thick silicone resin plate having, at the central portion thereof, air of which hole diameter is 0.2 mm to 2 mm. This bubble-containing resin plate is placed at a central portion of 2-cm thick silicone rubber and the maximum intensity of the ultrasound in the tank is measured. The result is 70 mW/cm². The acoustic diffusion layer 32 reduces the maximum value of ultrasound intensity to about 6% as compared with a case where no acoustic diffusion layer 32 is attached, and an ultrasonic beam can be diffused in a large area.

In the 1.5-MHz ultrasonic irradiation apparatus 10 of the period of 0.67 μs, PRF is 1 ms (1,000 Hz) and the duty factor of 20%. In an ultrasonic irradiation apparatus using this 1.5-MHz ultrasonic resonator, four stainless steel wire nets of which opening is 0.5 mm (50% with respect to the underwater wavelength λ) are stuck. The maximum value of ultrasound intensity emitted from ultrasonic resonator 20 as compared with case where it does not attach by using this acoustic diffusion layer is able to be reduced to 100 mW/cm² of about 10%, and ultrasonic beam is able to be diffused in large area. Isata is 93 mW/kg per body weight.

As the audible sound, the user's favorite music is played simultaneously. In this period, other knee therapeutic methods, such as intraarticular injection of hyaluronic acid and oral medication of chondroitin, are not performed. In a physical test of this patient after 50 weeks, 50-m running speed is 11 seconds, an average flight distance of 5 times of a soccer ball by instep kick is 33 m by the right foot and 20 m by the left foot, showing that the physical function has improved significantly. This athletic ability is kept after 1 year. An absolute value of knee extension muscular power after 1 year is 144 Nm, showing improvement about 15%.

Further, after about 1 year of investigation, hair restoration of the head to such a degree that close relatives can discriminate is observed. It has conventionally been reported that hair is restored by applying ultrasound stimulation to the head, and it is considered that the effect is exhibited by providing acoustic wave stimulation and LIPUS stimulation to the whole body. Further, the average unaided vision of both eyes of the subject at the start of investigation is 0.55, but has restored to 0.90 after completion of the investigation. It has also conventionally been reported that eyesight is restored by irradiating muscles around the eyes with ultrasound stimulation, and it is considered that the same effect is exhibited by applying low intensity ultrasound stimulation to the whole body.

As shown in a simple X-ray photograph of the front side of the right knee of this patient in a recumbent position under no load imaged January 2014 before the acoustic stimulation treatment is performed as illustrated in FIG. 10, medial joint space in the right knee has almost disappeared and a subchondral bone has hardened. Therefore, the patient has difficulty in walking and has a knee pain.

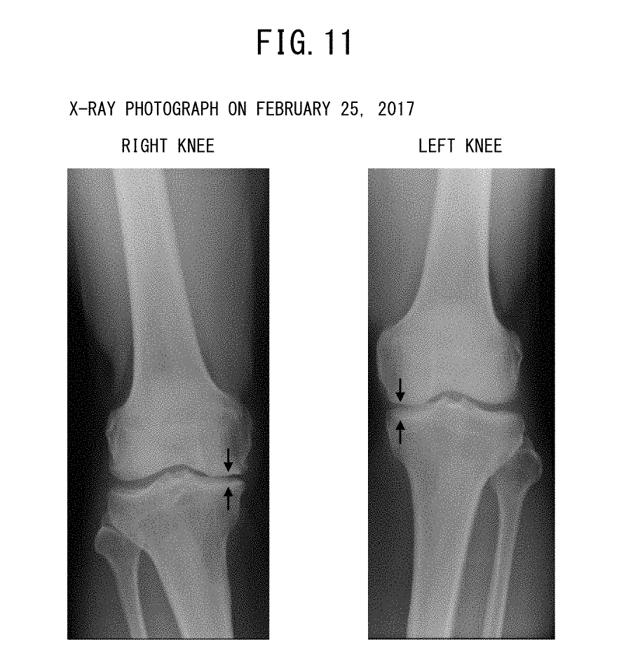

The patient is subject to 2 years treatment using the ultrasonic irradiation system and the ultrasonic irradiation method by the ultrasonic irradiation apparatus of the present invention. As a result, the state of the knee is improved as shown in the photograph of February 2017 in a recumbent position under no load as illustrated in FIG. 11. Regarding the state of the knee, as illustrated in FIG. 11, the medial joint space is increased. Also in an X-ray photograph in a single-leg standing position under load, it has confirmed that the joint space has increased slightly as compared with that of the photograph of the recumbent position under no load before the treatment. Regarding the left knee, no deformable change is observed both before and after the treatment, and the joint space is kept in a desirable state.

### EXAMPLE 2

Next, Example 2 of the ultrasonic irradiation apparatus, the ultrasonic irradiation system, and the method of the present invention will be described below. As Example 2, an example in which the present invention is applied to a 67.0-year-old male patient with pains in the right hip and outer right shin due to sciatic neuralgia caused by slipped disk whose body weight excluding the head is 50 kg will be described.

The patient is an amateur soccer player and has difficulty in walking and running due to low back pain after the age of 65. At the age of 66.0, the patient feels a sense of discomfort in the hip and the like and feels a pain in the outer right shin after walking about 10 minutes, so that breaks are necessary when he walks.

As treatment, two 0.33 MHz and 1.5 MHz ultrasonic irradiation apparatuses described above are used in a FRP bathtub for home use, and ultrasound irradiation is continued 10 min/times, 4 or 5 times/week, for 25 weeks. The patient jogs 1 or 2 times/week when 8 weeks elapse, but the part lower than the right shin is benumbed at the beginning. In this period, the patient only performs muscular training which is periodically and usually conducted. The patient starts training using a ball 1 time/week from the 9th week, and participates in a game from the 12th week, and conducts training 2 times/week (training and game). Although the patient feels a sense of discomfort in the right hip and the sole of the right foot is benumbed, the physical function has improved significantly. After 1 year, recurrence of the low back pain is not observed.

### EXAMPLE 3

Next, as Example 3 of the ultrasonic irradiation apparatus, the ultrasonic irradiation system, and the method of the present invention, an example in which the present invention is applied to a 67.0-year-old female patient with moderate low back pain whose body weight excluding the head is 36 kg will be described. The patient is a housewife and an elderly gymnast who has difficulty in walking and doing gymnastics for her low back pain after the age of 65. At the age of 66.0, walking speed of this patient is 100 m/min due to low back pain. Average grip strength of both hands is 22 kg. As treatment, the ultrasonic irradiation system 50 as illustrated in the schematic diagram of FIGS. 5A-5C in which foaming polyurethane having density of 0.1 g/cm³ and thickness of 0.6 cm is attached to a bottom surface and a side surface of an FRP bathtub for home use is used and acoustic wave irradiation is performed 10 min/time, 4 or 5 times/week, for 40 weeks.

As an ultrasonic resonator, a sodium potassium niobate-based lead-free ultrasound piezoelectric resonator of which diameter is 20 mm (area is 3.1 cm²), frequency is 4 MHz, and Isata is 300 mW/cm² is used. The ultrasonic irradiation apparatus 40 performs ultrasound irradiation at a period of 0.25 μs, PRF of 1000 Hz, and duty factor of 10% and at 26 mW/kg.

As an acoustic diffusion layer, as illustrated in FIG. 3, a member in which polystyrene foamed resin of a size of 1 mm to 3 mm and a density of 0.02 g/cm³ is dispersed into silicone rubber is used. Using the acoustic diffusion layer reduces the maximum strength of the ultrasonic beam of the ultrasonic resonator to 20% and diffuses the ultrasonic beam broadly. In this period, the patient performs only gymnastics training periodically and usually conducted. In a physical test of this patient after 40 weeks, walking speed is 120 m/min, average grip strength of both hands is 26 kg, showing that the physical function has improved.

The results described in Examples 1 to 3 show that the ultrasonic irradiation apparatus and the ultrasonic irradiation method of the present invention can significantly improve the physical function of elderly people by selecting conditions in which the resonance frequency of ultrasound of equal to or greater than 0.3 MHz and equal to or less than 5 MHz, and that the sound intensity Isata is in the range of 25 mW/kg to 1000 mW/kg, and applying the ultrasound to the whole body of the human body in the tank for substantially 25 weeks or more.

### DESCRIPTION OF REFERENCE NUMERALS

10, 30, 40, 64 ultrasonic irradiation apparatus

12 case

14 substrate

16 power supply

17 control unit

18 lead wire

19 high intensity ultrasound (HIUS)

20, 41, 42 ultrasonic resonator

21 low intensity ultrasound (LIUS)

22 acoustic matching layer

24, 32, 48 acoustic diffusion layer

26 portable electronic device

28 support base

44 loudspeaker

50, 60, 62, 76 ultrasonic irradiation system

52 tank

56 ultrasound reflector

65 ultrasonic oscillator

66 float

67 fixed part

68 connection line

72 inner wall

74 driving unit

1-21. (canceled)

22. An ultrasonic irradiation apparatus comprising:
an ultrasonic resonator capable of generating ultrasound;
a driving unit configured to drive the ultrasonic resonator;
a case holding the ultrasonic resonator and the driving unit;
an acoustic matching layer provided between the ultrasonic resonator and the case; and
an acoustic diffusion layer made of an ultrasound diffusing material configured to diffuse high intensity ultrasound emitted from the ultrasonic resonator and convert the high intensity ultrasound into low intensity ultrasound with low intensity per unit area and radiate in a large area;
wherein the ultrasonic resonator is a piezoelectric resonator, the ultrasound diffusing material of the acoustic diffusion layer is made of a metal material of an acoustic impedance of at least 40, and the acoustic diffusion layer is provided in at least one of inside of the acoustic matching layer, inside of the case, and outside of the case.

23. An ultrasonic irradiation apparatus comprising:
an ultrasonic resonator capable of generating ultrasound;
a driving unit configured to drive the ultrasonic resonator;
a case holding the ultrasonic resonator and the driving unit;
an acoustic matching layer provided between the ultrasonic resonator and the case; and
an acoustic diffusion layer made of an ultrasound diffusing material configured to diffuse high intensity ultrasound emitted from the ultrasonic resonator and convert the high intensity ultrasound into low intensity ultrasound with low intensity per unit area and radiate in a large area;
wherein the ultrasonic resonator is a piezoelectric resonator, the ultrasound diffusing material of the acoustic diffusion layer is made of a metal material, and is made of a porous wire net of which opening is λ-λ/10 with respect to an underwater wavelength λ of used ultrasound.

24. An ultrasonic irradiation apparatus comprising:
an ultrasonic resonator capable of generating ultrasound;
a driving unit configured to drive the ultrasonic resonator;
a case holding the ultrasonic resonator and the driving unit;
an acoustic matching layer provided between the ultrasonic resonator and the case; and
an acoustic diffusion layer made of an ultrasound diffusing material configured to diffuse high intensity ultrasound emitted from the ultrasonic resonator and convert the high intensity ultrasound into low intensity ultrasound with low intensity per unit area and radiate in a large area;
wherein the ultrasonic resonator is a piezoelectric resonator, and the ultrasound diffusing material of the acoustic diffusion layer is made of foamed resin containing 90 to 99 volume % of air bubbles and gas.

25. The ultrasonic irradiation apparatus according to claim 22, wherein the ultrasonic resonator is made of a lead-free piezoelectric material.

26. The ultrasonic irradiation apparatus according to claim 23, wherein the ultrasonic resonator is made of a lead-free piezoelectric material.

27. The ultrasonic irradiation apparatus according to claim 24, wherein the ultrasonic resonator is made of a lead-free piezoelectric material.

28. The ultrasonic irradiation apparatus according to claim 22, wherein positions at which lead wires of the piezoelectric resonator and a power supply thereof are drawn are separated from the ultrasonic resonator in the case and located at an upper part of the case when the apparatus is in use.

29. The ultrasonic irradiation apparatus according to claim 23, wherein positions at which lead wires of the piezoelectric resonator and a power supply thereof are drawn are separated from the ultrasonic resonator in the case and located at an upper part of the case when the apparatus is in use.

30. The ultrasonic irradiation apparatus according to claim 24, wherein positions at which lead wires of the piezoelectric resonator and a power supply thereof are drawn are separated from the ultrasonic resonator in the case and located at an upper part of the case when the apparatus is in use.

31. An ultrasonic irradiation apparatus comprising:
an ultrasonic resonator capable of generating ultrasound;
a driving unit configured to drive the ultrasonic resonator;
a case holding the ultrasonic resonator and the driving unit; and
an acoustic matching layer provided between the ultrasonic resonator and the case;
wherein the ultrasonic resonator having at least one of two frequencies and two pulse repetition frequencies is placed in the case, and these continuously and automatically generate a plurality of ultrasounds when driven by the driving unit.

32. The ultrasonic irradiation apparatus according to claim 22, wherein:
the driving unit including a power supply of the ultrasonic resonator and an ultrasonic oscillator including the ultrasonic resonator and the acoustic diffusion layer are divided, and the divided ultrasonic oscillator is waterproof and is provided to be electrically connectable to the driving unit, and
the ultrasonic oscillator is carried by a holding member which is floatable on a water surface, the divided ultrasonic oscillator is connected to the driving unit, so that the ultrasonic oscillator is floatable and movable under the water surface.

33. The ultrasonic irradiation apparatus according to claim 23, wherein:
the driving unit including a power supply of the ultrasonic resonator and an ultrasonic oscillator including the ultrasonic resonator and the acoustic diffusion layer are divided, and the divided ultrasonic oscillator is waterproof and is provided to be electrically connectable to the driving unit, and
the ultrasonic oscillator is carried by a holding member which is floatable on a water surface, the divided ultrasonic oscillator is connected to the driving unit, so that the ultrasonic oscillator is floatable and movable under the water surface.

34. The ultrasonic irradiation apparatus according to claim 24, wherein:
the driving unit including a power supply of the ultrasonic resonator and an ultrasonic oscillator including the ultrasonic resonator and the acoustic diffusion layer are divided, and the divided ultrasonic oscillator is waterproof and is provided to be electrically connectable to the driving unit, and
the ultrasonic oscillator is carried by a holding member which is floatable on a water surface, the divided ultrasonic oscillator is connected to the driving unit, so that the ultrasonic oscillator is floatable and movable under the water surface.

35. The ultrasonic irradiation apparatus according to claim 22, wherein the acoustic matching layer of at least two types of materials is included, a thickness of the acoustic matching layer is multiples of ¼ of an underwater wavelength λ of the used ultrasound, and a contour shape of the acoustic matching layer is 120% to 200% of the ultrasonic resonator in magnitude in a project area.

36. The ultrasonic irradiation apparatus according to claim 23, wherein the acoustic matching layer of at least two types of materials is included, a thickness of the acoustic matching layer is multiples of ¼ of an underwater wavelength λ of the used ultrasound, and a contour shape of the acoustic matching layer is 120% to 200% of the ultrasonic resonator in magnitude in a project area.

37. The ultrasonic irradiation apparatus according to claim 24, wherein the acoustic matching layer of at least two types of materials is included, a thickness of the acoustic matching layer is multiples of ¼ of an underwater wavelength λ of the used ultrasound, and a contour shape of the acoustic matching layer is 120% to 200% of the ultrasonic resonator in magnitude in a project area.

38. The ultrasonic irradiation apparatus according to claim 22, wherein a part of the acoustic matching layer is a transparent organic material which constitutes the case, and a thickness thereof is multiples ¼ of λ.

39. The ultrasonic irradiation apparatus according to claim 23, wherein a part of the acoustic matching layer is a transparent organic material which constitutes the case, and a thickness thereof is multiples ¼ of λ.

40. The ultrasonic irradiation apparatus according to claim 24, wherein a part of the acoustic matching layer is a transparent organic material which constitutes the case, and a thickness thereof is multiples ¼ of λ.

41. The ultrasonic irradiation apparatus according to claim 22, wherein a fundamental wave frequency of each of the ultrasonic resonators is in a range of 0.3 MHz to 5 MHz, a plurality of acoustic matching layers are attached to the piezoelectric resonator of each ultrasonic resonator, the ultrasounds of a plurality of resonance frequencies are made to generate then, and the ultrasounds of at least two different frequencies are made to continuously and automatically generate by a single piezoelectric resonator.

42. The ultrasonic irradiation apparatus according to claim 23, wherein a fundamental wave frequency of each of the ultrasonic resonators is in a range of 0.3 MHz to 5 MHz, a plurality of acoustic matching layers are attached to the piezoelectric resonator of each ultrasonic resonator, the ultrasounds of a plurality of resonance frequencies are made to generate then, and the ultrasounds of at least two different frequencies are made to continuously and automatically generate by a single piezoelectric resonator.

43. The ultrasonic irradiation apparatus according to claim 24, wherein a fundamental wave frequency of each of the ultrasonic resonators is in a range of 0.3 MHz to 5 MHz, a plurality of acoustic matching layers are attached to the piezoelectric resonator of each ultrasonic resonator, the ultrasounds of a plurality of resonance frequencies are made to generate then, and the ultrasounds of at least two different frequencies are made to continuously and automatically generate by a single piezoelectric resonator.

44. The ultrasonic irradiation apparatus according to claim 22, wherein at least two ultrasonic resonators are placed on a side surface of the case which is shaped as a pyramid, a cone, or a sphere, and each ultrasound radiation surface cross at an angle range of 60° to 200°.

45. The ultrasonic irradiation apparatus according to claim 23, wherein at least two ultrasonic resonators are placed on a side surface of the case which is shaped as a pyramid, a cone, or a sphere, and each ultrasound radiation surface cross at an angle range of 60° to 200°.

46. The ultrasonic irradiation apparatus according to claim 24, wherein at least two ultrasonic resonators are placed on a side surface of the case which is shaped as a pyramid, a cone, or a sphere, and each ultrasound radiation surface cross at an angle range of 60° to 200°.

47. The ultrasonic irradiation apparatus according to claim 22, wherein the ultrasound is a pulse wave, a repetition frequency (PRF) of the pulse wave is 1000 Hz to 0.5 Hz, and a duty factor is 10% to 60%, and the PRF continuously and automatically generates at least two selected from 1000 Hz to 33 Hz (period of 1 ms to 3 ms), 50 Hz to 25 Hz (period of 20 ms to 40 ms), and 2 Hz to 0.5 Hz (period of 500 ms to 2000 ms).

48. The ultrasonic irradiation apparatus according to claim 22, further comprising a removable portable electronic device or another apparatus which generates music of audible sound.

49. The ultrasonic irradiation apparatus according to claim 23, further comprising a removable portable electronic device or another apparatus which generates music of audible sound.

50. The ultrasonic irradiation apparatus according to claim 24, further comprising a removable portable electronic device or another apparatus which generates music of audible sound.

51. An ultrasonic irradiation system comprising the ultrasonic irradiation apparatus according to claim 22, wherein the ultrasonic irradiation apparatus is provided in at least one of a tank capable of receiving human or mammals and filled with water, on a water surface or in the water of the tank, and the ultrasonic irradiation apparatus is provided in the water in the tank capable of radiating ultrasound.

52. An ultrasonic irradiation system comprising the ultrasonic irradiation apparatus according to claim 23, wherein the ultrasonic irradiation apparatus is provided in at least one of a tank capable of receiving human or mammals and filled with water, on a water surface or in the water of the tank, and the ultrasonic irradiation apparatus is provided in the water in the tank capable of radiating ultrasound.

53. An ultrasonic irradiation system comprising the ultrasonic irradiation apparatus according to claim 24, wherein the ultrasonic irradiation apparatus is provided in at least one of a tank capable of receiving human or mammals and filled with water, on a water surface or in the water of the tank, and the ultrasonic irradiation apparatus is provided in the water in the tank capable of radiating ultrasound.

54. The ultrasonic irradiation system according to claim 51, wherein an ultrasound reflector with at least 80% of ultrasonic reflectance for reflecting and diffusing ultrasound is attached to at least 80% of a surface area of an inner wall surface of the tank.

55. The ultrasonic irradiation system according to claim 52, wherein an ultrasound reflector with at least 80% of ultrasonic reflectance for reflecting and diffusing ultrasound is attached to at least 80% of a surface area of an inner wall surface of the tank.

56. The ultrasonic irradiation system according to claim 53, wherein an ultrasound reflector with at least 80% of ultrasonic reflectance for reflecting and diffusing ultrasound is attached to at least 80% of a surface area of an inner wall surface of the tank.

57. The ultrasonic irradiation system according to claim 54, wherein the ultrasound reflector is a composite material, density of a tank inner surface material of a tank outer surface material thereof is 0.01 g/cm3 to 0.1 g/cm3, and is made of a foamed material which is an organic material containing gas.

58. The ultrasonic irradiation system according to claim 55, wherein the ultrasound reflector is a composite material, density of a tank inner surface material of a tank outer surface material thereof is 0.01 g/cm3 to 0.1 g/cm3, and is made of a foamed material which is an organic material containing gas.

59. The ultrasonic irradiation system according to claim 56, wherein the ultrasound reflector is a composite material, density of a tank inner surface material of a tank outer surface material thereof is 0.01 g/cm3 to 0.1 g/cm3, and is made of a foamed material which is an organic material containing gas.

60. The ultrasonic irradiation system according to claim 57, wherein the ultrasound reflector is a sheet, a waterproof sheet is provided on a surface of the sheet, and a foamed organic material containing 90 to 99 volume % of gas inside of the sheet.

61. The ultrasonic irradiation system according to claim 58, wherein the ultrasound reflector is a sheet, a waterproof sheet is provided on a surface of the sheet, and a foamed organic material containing 90 to 99 volume % of gas inside of the sheet.

62. The ultrasonic irradiation system according to claim 59, wherein the ultrasound reflector is a sheet, a waterproof sheet is provided on a surface of the sheet, and a foamed organic material containing 90 to 99 volume of gas inside of the sheet.

63. The ultrasonic irradiation system according to claim 51, further comprising a bubble generator configured to discharge air bubbles of 0.01 mm to 10 mm in diameter into water.

64. The ultrasonic irradiation system according to claim 52, further comprising a bubble generator configured to discharge air bubbles of 0.01 mm to 10 mm in diameter into water.

65. The ultrasonic irradiation system according to claim 53, further comprising a bubble generator configured to discharge air bubbles of 0.01 mm to 10 mm in diameter into water.

66. An ultrasonic irradiation method comprising:
placing the ultrasonic irradiation apparatus according to claim 22, or a part thereof which includes the ultrasonic resonator in a tank,
making the ultrasonic resonator floating and moving on water,
making the ultrasonic resonator continuously and automatically generate multi-ultrasound with different frequencies and PRFs,
making the ultrasound reflected on an inner wall and a water surface of the tank, and
providing stimulation by low intensity multi-ultrasound to the whole body or a part of the body of human or mammals in the water.

67. An ultrasonic irradiation method comprising:
placing the ultrasonic irradiation apparatus according to claim 23, or a part thereof which includes the ultrasonic resonator in a tank,
making the ultrasonic resonator floating and moving on water,
making the ultrasonic resonator continuously and automatically generate multi-ultrasound with different frequencies and PRFs,
making the ultrasound reflected on an inner wall and a water surface of the tank, and
providing stimulation by low intensity multi-ultrasound to the whole body or a part of the body of human or mammals in the water.

68. An ultrasonic irradiation method comprising:
placing the ultrasonic irradiation apparatus according to claim 24, or a part thereof which includes the ultrasonic resonator in a tank,
making the ultrasonic resonator floating and moving on water,
making the ultrasonic resonator continuously and automatically generate multi-ultrasound with different frequencies and PRFs,
making the ultrasound reflected on an inner wall and a water surface of the tank, and
providing stimulation by low intensity multi-ultrasound to the whole body or a part of the body of human or mammals in the water.

69. The ultrasonic irradiation method according to claim 66, wherein intensity spatial average temporal average (Isata) of 25 mW/kg to 1000 mW/kg per body weight of an ultrasound irradiation part is radiated to human or mammals in the tank.

70. The ultrasonic irradiation method according to claim 67, wherein intensity spatial average temporal average (Isata) of 25 mW/kg to 1000 mW/kg per body weight of an ultrasound irradiation part is radiated to human or mammals in the tank.

71. The ultrasonic irradiation method according to claim 68, wherein intensity spatial average temporal average (Isata) of 25 mW/kg to 1000 mW/kg per body weight of an ultrasound irradiation part is radiated to human or mammals in the tank.

72. The ultrasonic irradiation method according to claim 66, wherein a temperature of water in the tank is set to be 37° C. to 42° C., each ultrasound of different frequencies and PRFs is continuously and automatically radiated from the ultrasonic resonator in series in a certain period of time, the ultrasound is applied to the whole body or a part of the body excluding head of human or mammals in a tank 10 to 60 min/day, 2 to 7 days/week, for 2 to 50 weeks.

73. The ultrasonic irradiation method according to claim 67, wherein a temperature of water in the tank is set to be 37° C. to 42° C., each ultrasound of different frequencies and PRFs is continuously and automatically radiated from the ultrasonic resonator in series in a certain period of time, the ultrasound is applied to the whole body or a part of the body excluding head of human or mammals in a tank 10 to 60 min/day, 2 to 7 days/week, for 2 to 50 weeks.

74. The ultrasonic irradiation method according to claim 68, wherein a temperature of water in the tank is set to be 37° C. to 42° C., each ultrasound of different frequencies and PRFs is continuously and automatically radiated from the ultrasonic resonator in series in a certain period of time, the ultrasound is applied to the whole body or a part of the body excluding head of human or mammals in a tank 10 to 60 min/day, 2 to 7 days/week, for 2 to 50 weeks.
